(19) United States
(12) Patent Application Publication
(10) Pub. No.: US 2002/0055753 A1
(43) Pub. Date: May 9, 2002

(54) CORNEAL IMPLANT METHODS AND PLIABLE IMPLANT THEREFOR

(76) Inventor: THOMAS A. SILVESTRINI, ALAMO, CA (US)

Correspondence Address: Antoinette F. Konski, McCutchen Doyle Brown & Enersen, Three Embarcadero Center, Suite 1800, San Francisco, CA, 94111-4067, US

(21) Appl. No.: 08/993,946

(22) Filed: Dec. 18, 1997

(51) Int. Cl. A61F 2/14

(52) U.S. Cl. 606/166; 623/5.12; 623/906

(57) ABSTRACT

This invention involves an improved surgical method and associated apparatus for correcting refractive defects of the vision, using an intracorneal implant. A small radial incision is made in the periphery of the cornea, near the limbus and a blunt spatula is used to separate the lamellae of the corneal stroma. A circular interlamellar pathway through the stroma is formed using either a single 360 degree blunt, arc-shaped dissector tool or a pair of clockwise and counterclockwise 180-200 degree dissector tools. The circular pathway created defines the margin or outer boundary of an intracorneal channel that will be formed. The intracorneal channel is then expanded radially inward in a controlled stepwise fashion to widen the channel or to create an intracorneal pocket. This is done by introducing a dissector tool with a side leg into the incision and moving the dissector tool in an arc-shaped path to widen the intracorneal channel. A single 360 degree dissector tool or a pair of clockwise and counterclockwise 180-200 degree dissector tools can be used. Dissector tools with progressively longer side legs are used to expand the channel until the desired width is achieved or until a complete intracorneal pocket is created. An intracorneal implant, which may be a split ring, segmented ring or continuous ring intracorneal implant or an intracorneal lens implant, is inserted into the channel or pocket and the incision is closed. The intracorneal implant is positioned remotely from the incision so that less stress is exerted on the incision during healing. The surgical apparatus, including the blunt, arc-shaped dissector tools and the side-leg dissector tools can be designed to be operated manually or with a vacuum centering guide which allows careful and precise control over the intracorneal channel created.

### BACKGROUND OF THE INVENTION

The present invention relates generally to an improved surgical method and apparatus for correcting defects in vision. More particularly, it relates to a method and apparatus for surgically implanting an intracorneal implant for correcting various refractive defects of the vision.

In order to more fully understand the present invention it is important to have an understanding of the function of the eye and the various defects, which can effect the vision. Ametropia, which is responsible for various refractive defects of the vision, is caused by a discrepancy between the refractive power of the eye and the dimensions of the eye, such that images are not brought into proper focus on the retina. Forms of ametropia include myopia, hyperopia and astigmatism. In the normal or emmetropic eye, light rays from a distant object which enter the eye parallel to the optical axis are focused directly on the retina resulting in a clear image of distant objects. The light rays are focused by the combined refractive power of the cornea and the crystalline lens of the eye. The light rays are first refracted at the anterior surface of the cornea, then refracted again at each interface between the cornea, the aqueous humor, the crystalline lens and the vitreous humor. Since the greatest difference in refractive index is at the interface between the cornea and the air, most of the refraction occurs at the anterior surface of the cornea. Light rays from near objects reach the eye at a divergent angle. The diverging light rays would normally be focused at a point behind the retina, resulting in an unfocused image of near objects. However, the eye brings the image into clear focus by accommodation of the crystalline lens through the action of the ciliary muscles, which surround the crystalline lens.

Accommodation results in a thickening of the crystalline lens which increases its degree of curvature and therefore its refractive power so that the image is brought to a sharp focus on the retina. The amplitude of accommodation of the crystalline lens determines how close objects can be and still be focused sharply on the retina. The closest distance at which the eye can still bring an object into focus is called the near point of distinct vision.

Myopia or nearsightedness is a form of ametropia caused by a mismatch between the refractive power of the eye and the dimensions of the eye that results in light rays entering the eye parallel to the optical axis being focused in front of the retina. Axial myopia is caused by the anteroposterior axis of the eye being too short, while curvature myopia is caused by excessive convexity of the refractive surfaces of the cornea and/or the lens. In the myopic eye, light rays from a distant object which enter the eye parallel to the optical axis are focused at a point in front of the retina. By the time the light rays have reached the retina, they have already diverged somewhat, resulting in an unfocused image of distant objects. On the other hand, the diverging light rays from near objects can be brought into sharp focus on the retina, with little or no accommodation of the crystalline lens, depending on the degree of myopia. With full accommodation of the crystalline lens, the myopic eye can focus light rays from objects that are very close to the eye. The near point of distinct vision is very close to the eye, hence the term nearsightedness. Nearsightedness has traditionally been treated with negative power corrective lenses, either with spectacles or contact lenses, which diverge the light rays somewhat before they reach the eye, resulting in normal, clear vision at all distances.

Hyperopia or farsightedness is a form of ametropia caused by a mismatch between the refractive power of the eye and the dimensions of the eye that results in light rays entering the eye parallel to the optical axis being focused behind the retina. Axial hyperopia is caused by shortness of the anteroposterior axis of the eye, while curvature hyperopia is caused by insufficient convexity of the refractive surfaces of the cornea and/or the lens. In the hyperopic eye, light rays from a distant object which enter the eye parallel to the optical axis are focused at a point behind the retina, which would normally result in an unfocused image of distant objects. However, with accommodation of the crystalline lens, the eye can bring the image into sharp focus on the retina for clear vision of distant objects. For near objects, the hyperopic eye focuses the diverging light rays which enter the eye at a point very far behind the retina. The hyperopic eye attempts to bring the image into focus through accommodation of the crystalline lens. However, because there is a limit to the amplitude of accommodation possible for the crystalline lens, the point of focus for near objects still falls behind the retina, resulting in an unfocused image. The near point of distinct vision that can be accomplished through full accommodation of the crystalline lens is farther removed from the eye, hence the term farsightedness. Farsightedness has traditionally been treated with positive power corrective lenses, either with spectacles or contact lenses, which converge the light rays somewhat before they reach the eye, resulting in normal, clear vision at all distances. Alternatively, because the eye can accommodate sufficiently for distant vision without corrective lenses, moderate amounts of hyperopia are sometimes treated with “reading glasses” which are only needed for viewing objects closer than the near point of distinct vision.

Astigmatism is a form of ametropia caused by the radius of curvature of the refractive surfaces of the cornea and/or the lens of the eye in one plane being longer or shorter than the radius of curvature in a plane at right angles to it. As a result, rays of light entering the eye are not focused at a sharp point on the retina, but are spread over a diffuse area. Astigmatism can occur in combination with myopia, hyperopia or presbyopia. Astigmatism is traditionally treated with toric corrective lenses that have greater or lesser refractive power in one plane than in the plane at right angles to it. A negative power correction for myopia or a positive power correction for hyperopia can be superimposed on the toric correction for astigmatism. Astigmatism is usually corrected with spectacles, however some degree of success has been achieved for correcting modest amounts of astigmatism with toric contact lenses.

In addition to the traditional approach of correcting ametropia with corrective lenses, various surgical methods for vision correction are also known. Recognized surgical methods include radial keratotomy, exemplified by U.S. Pat. No. 4,688,570 granted to Kramer et al., entitled Opthalmologic Surgical Instrument, and U.S. Pat. No. 4,815,463 granted to Hana, entitled Surgical Apparatus for Radial Keratotomy, and photorefractive keratectomy, exemplified by U.S. Pat. No. 4,941,093 granted to Marshall et al., entitled Surface Erosion Using Lasers, and U.S. Pat. No. 5,163,934 granted to Munnerlyn, entitled Photorefractive Keratectomy. In radial keratotomy and photorefractive keratectomy, the cornea of the eye is reshaped by cutting or by laser ablation to correct vision defects. These surgical approaches have significant drawbacks in that both methods involve substantial trauma to the cornea of the eye from multiple incisions or laser ablations in or near the optical zone of the cornea. Such trauma can result in the formation of scar tissue, which, if it extends into the optical zone of the cornea, can interfere with the patient's vision. Also, in a small percentage of cases, the results of the surgery are unsatisfactory and can even cause a deterioration of the patient's vision instead of an improvement. Unfortunately, the effects of radial keratotomy and photorefractive keratectomy are irreversible so the patient must accept the outcome of the surgery if it is unsuccessful.

Another surgical approach for treating refractive defects of the vision involves the use of corrective implants surgically implanted into the cornea of the eye. One variant of this surgical approach is to implant a corrective lens directly into the optical zone of the cornea to correct the patient's vision. A second variant of this surgical approach involves the use of intracorneal implants for modifying the actual curvature of the corneal surface.

For example, U.S. Pat. No. 4,655,774 granted to Choyce for an Intra-Corneal Implant for Correction of Aniridia describes a method for implanting an artificial iris with an optional corrective lens within the cornea of the eye for correcting vision defects. The surgical method described involves making an incision into the cornea, creating a pocket within the cornea using a curved dissecting instrument, inserting the implant into the pocket and closing the incision. The method requires an incision at least as large as the diameter of the rigid implant (estimated to be about 6-8 mm) and the pocket forming step does not provide positive control of the margins of the pocket formed. These aspects of the surgical procedure may inhibit healing of the cornea after implantation of the device.

U.S. Pat. No. 5,196,026 granted to Barret et al. for a Method of Implanting Corneal Inlay Lenses Smaller Than the Optic Zone describes a surgical method that involves making an incision near the edge of the cornea the size of the lens to be inserted and creating a pocket to the center of the cornea using a spatula. A circular or ring-shaped lens 2-4 mm in diameter is inserted into the pocket. This method allows a smaller incision than Choyce, but only because the actual implant is smaller. Using this method for larger implants to affect the entire optical zone would naturally require a larger, more traumatic incision. This method also does not provide positive control of the margins of the pocket formed.

U.K. patent GB 2,095,119 granted to Tennant et al. for a Circular Keratotomy with Insert for Myopia Correction describes a surgical method wherein the epithelial layer of the cornea is removed and the optical zone is circumscribed with a circular groove which causes the cornea to flatten. A circular insert is positioned within the groove to maintain the space while scar tissue grows to cover the insert and the epithelium regrows over the corneal surface. This method involves considerable trauma to the eye in that it requires removal of the epithelial layer and a large circular incision around the optical zone. Because of the amount of scar tissue produced, this procedure would not be reversible without substantial trauma to the corneal tissues.

U.S. Pat. No. 4,976,719 granted to Siepser for a Device used to Change Corneal Curvature describes a similar ring-shaped corneal implant that includes the improvement of a turnbuckle connector which allows the size of the implant to be adjusted in order to correct for myopia or hyperopia. The surgical procedure for implanting the ring-shaped implant avoids the need for a circular incision by inserting one end of the ring through a puncture in the cornea and advancing it in a circular path between the corneal layers. However, a 4 to 5 mm incision is still required for manipulation of the turnbuckle. In addition, there is no positive control of the path of the wire ring as it is advanced through the corneal tissue.

U.S. Pat. No. 5,391,201 granted to Barret et al. for a Method of Using a Corneal Ring Inlay describes a surgical method for implanting a continuous ring into the cornea that involves either a peripheral incision in the cornea followed by undermining the cornea in a circular fashion or slicing the top of the cornea off completely. Either of these surgical approaches is highly traumatic to the corneal tissue and would inhibit healing of the cornea. The consequent scarring would likely make this procedure irreversible.

U.S. Pat. No. 4,452,235 granted to Reynolds for a Method for Corneal Curvature Adjustment describes a surgical method for implanting a split ring within the cornea for correcting vision defects. A 1 mm incision is made in the cornea and a circular dissecting tool is used to create a circular path within the cornea back to the incision point. One end of the split ring is connected to the dissecting tool and the tool is backed out, pulling the split ring in behind it. Once the split ring is inside the cornea, it is detached from the tool and the diameter of the ring is adjusted to correct the patient's vision, then the ends of the ring are fixed together. This method creates a very small incision in the cornea which promotes healing. However, the incision is directly over the split ring implant, which may result in stress on the incision that could interfere with healing or increase scar tissue formation.

U.S. Pat. No. 5,403,335 granted to Loomis et al. for a Corneal Vacuum Centering Guide and Dissector describes a method and associated apparatus for creating a circular interlamellar pathway within the corneal stroma of the eye for implanting an intracorneal ring to correct vision defects. The apparatus carefully controls the creation of the interlamellar pathway by using a vacuum centering guide to hold the cornea and guide the dissecting tool along a precise path. In this method also, the small incision is positioned directly over the path of the split ring implant.

Because of the disadvantages of these various prior art approaches, it is desirable to provide an improved surgical method and associated apparatus for correcting refractive defects of the eye using an intracorneal implant. It is desirable that such a method provide a permanent, but reversible, correction of vision defects without substantial trauma to the corneal tissue. To this end, it is desirable to minimize the extent of any incisions into the cornea and to isolate the incision from any stress caused by the presence of the intracorneal implant in order to promote healing and reduce scar tissue formation. It is also desirable to provide a surgical method with the flexibility to produce either a circular interlamellar pathway for implanting a split ring or segmented ring intracorneal implant or an intracorneal pocket for implanting a continuous ring intracorneal implant or intracorneal lens implant. When operated in either mode, the method and associated apparatus should allow careful control over the margins of the interlamellar pathway or intracorneal pocket which is formed.

### SUMMARY OF THE INVENTION

In keeping with the foregoing discussion, the present invention provides an improved surgical method and associated apparatus for correcting refractive defects of the vision, including myopia, hyperopia and astigmatism, using an intracorneal implant. The method may involve creating a widened channel or pocket through a single incision and inserting a suitable implant through the same incision.

According to one aspect of the method, a small incision is made in the periphery of the cornea, near the limbus. A small, blunt spatula may be used to make an initial separation between the lamellar layers of the corneal stroma. A blunt, arc-shaped dissector tool is then used to create a circular interlamellar pathway through the stroma. A circular dissector tool which subtends an arc of about 360 degrees may be used to create the circular pathway in a single operation or a pair of clockwise and counterclockwise semicircular dissector tools, each subtending an arc of approximately 180-200 degrees, may be used to create the circular pathway in two steps.

In one aspect, the circular pathway created by the dissector defines the margin or outer boundary of the intracorneal channel that will be formed. Defining the boundary of the intracorneal channel in a controlled and predictable manner this way creates a smooth outer margin which promotes healing, and it avoids inadvertently extending the intracorneal channel into the limbus which could lead to ingrowth of blood vessels into the cornea that would impair the patient's vision.

The intracorneal channel is then expanded radially inward in a controlled stepwise fashion to widen the channel or to create an intracorneal pocket. This is done by introducing a channel-widening dissector tool with a side leg into the incision and moving the channel-widening dissector tool in an arc-shaped path to widen the intracorneal channel. Once again, this can be done with a circular channel-widening dissector tool or a pair of clockwise and counterclockwise 180-200 degree semicircular channel-widening dissector tools. Channel-widening dissector tools with progressively longer side legs are used to expand the channel until the desired width is achieved or until a complete intracorneal pocket is created.

Once the intracorneal channel or pocket is completed, the appropriately shaped intracorneal implant, which may be a split ring, segmented ring or continuous ring intracorneal implant or an intracorneal lens implant, is inserted into the channel and the incision is closed. The widening of the intracorneal channel allows the intracorneal implant to be positioned remotely from the incision so that no unnecessary stress is exerted on the incision during healing.

Another aspect of the invention involves a pliable continuous ring implant. The continuous ring implant is constructed for insertion into an intracorneal pocket in a folded, compressed, or stretched state. The continuous ring implant may be inserted using standard forceps or forceps having end effectors specifically adapted to hold the ring in a compressed or folded state for insertion. To further aid insertion, the folded ring may be assembled into a tubular element which is then at least partially inserted into the pocket through the incision. The ring is then pulled from the tube using a hook shaped instrument. Preferably, the tubular element is arc-shaped. The continuous ring implant may also be inserted in a stretched state. This may be accomplished by assembling the ring on an insertion instrument having appropriately spaced protrusions.

The small size of the initial incision and the isolation of the incision from any stress due to the presence of the implant will reduce the likelihood of scar tissue formation, which will contribute to the positive results of the vision correction surgery, and, in the event that the results are unsatisfactory or if there are other complications, will contribute to the reversibility of the procedure.

The apparatus for performing the improved surgical method, including the arc-shaped dissector tool or tools and the side-leg channel-widening and pocket-forming dissector tools can be designed to be manually operated or, quite advantageously, they can be adapted to operate in cooperation with a vacuum centering guide. This combination allows careful and precise control over the intracorneal channel created.

These and other advantages of the present invention will become apparent to those skilled in the art from consideration of the following detailed description of the invention along with the accompanying drawings.

### BRIEF DESCRIPTION OF THE DRAWINGS

FIG. 1 is a flow chart showing the steps A-O of the surgical method of the present invention.

FIG. 2 is a diagram showing a circumferential incision according to the principles of the present invention.

FIGS. 3A-3B is a sectional view taken along line 3-3 of FIG. 1.

FIG. 3C is a sectional view of an oblique incision according to principles of the present invention. FIG. 4A is a front view of the pocketing tool constructed according to the principles of the present invention.

FIG. 4B is a detailed view of the tip section of the pocketing tool of FIG. 4A.

FIG. 4C is a side view of the tip section of FIG. 4A.

FIG. 4D illustrates the operation of the pocketing tool of FIGS. 4A-4C.

FIG. 5 shows a plan view of a spreader according to the present invention.

FIG. 5A shows a partial view of the spreader of FIG. 5, starting from cut lines A-A.

FIG. 5B is a partial view similar to that of FIG. 8A, but rotated 90 degrees.

FIG. 5C is a sectional view taken along lines C-C in FIG. 5B.

FIG. 5D is a sectional view taken along lines D-D in FIG. 5B.

FIG. 5E is a magnified view of the tip of FIG. 5A starting from cut lines E-E.

FIG. 5F is a modification of the tip shown in FIG. 8A.

FIG. 6A is a front perspective view of the vacuum centering device according to the principles of the present invention.

FIG. 6B is a perspective view of the vacuum centering device as viewed from approximately along line 6B-6B as shown in FIG. 1.

FIGS. 7A-7C show a circular dissector tool for creating a circular interlamellar pathway through the corneal stroma.

FIGS. 8A-8C show a clockwise semicircular dissector tool for creating a circular interlamellar pathway.

FIGS. 9A-9C show a counterclockwise semicircular dissector tool for creating a circular interlamellar pathway.

FIGS. 10A-10C show a circular channel-widening dissector tool with a side leg for widening the intracorneal channel. FIGS. 11A-11C show a clockwise semicircular channel-widening dissector tool with a side leg for widening the intracorneal channel.

FIGS. 12A-12C show a counterclockwise semicircular channel-widening dissector tool with a side leg for widening the intracorneal channel.

FIGS. 13A-13C show a circular pocket-forming dissector tool with a side leg for creating an intracorneal pocket.

FIGS. 14A-14C show a clockwise semicircular pocket-forming dissector tool with a side leg for creating an intracorneal pocket.

FIGS. 15A-15C show a counterclockwise semicircular pocket-forming dissector tool with a side leg for creating an intracorneal pocket.

FIG. 16A is a plan view showing a method of inserting a continuous ring implant.

FIG. 16B is a plan view showing an alternate method of inserting a continuous ring implant.

FIG. 16C is cross-sectional view taken along the line 16C-16C of FIG. 16B.

FIG. 17 is a partial plan view showing a specialized end effector for inserting a continuous ring implant.

FIG. 18 is a perspective view of a tool for inserting a continuous ring implant.

FIG. 19 shows an illustrative implant in partial cross-section.

FIG. 20A is a cross-sectional illustration of a folded implant within an introducer barrel.

FIG. 20B is a cross-sectional illustration of a rolled implant within an introducer barrel.

### DETAILED DESCRIPTION OF THE INVENTION

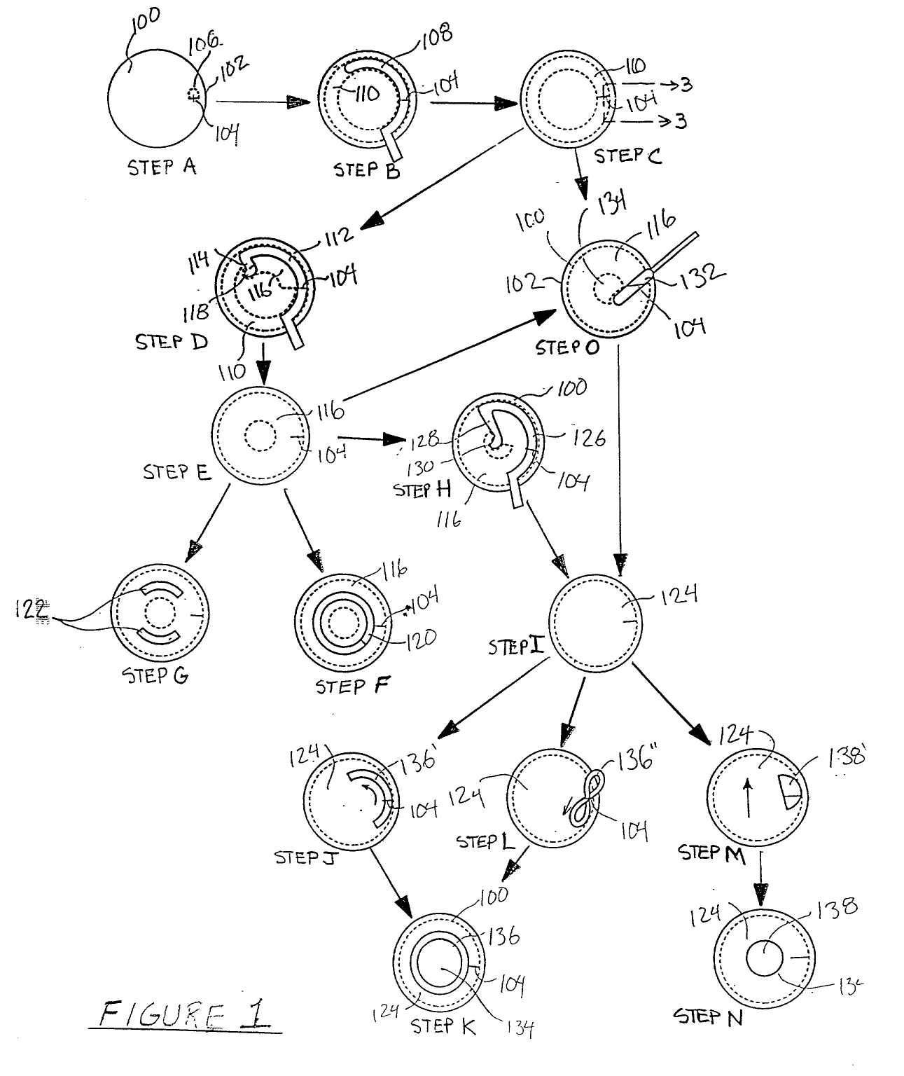

FIG. 1 is a flow chart illustrating steps A-O of the improved surgical methods of the present invention for correcting refractive defects of the vision using an intracorneal implant. According to the principles of the present invention, the surgical methods generally involve making a small incision, creating an initial substantially circular intrastromal corneal channel using various dissector instruments, widening the channel as desired, and positioning an appropriate insert for correction of any number of vision defects.

Certain types of inserts, such as split rings or intracorneal segments or the like, may be inserted into a widened channel such that the insert is positioned so as not to be directly under the incision. In addition, the channel may be further widened towards the center until the full area of the cornea inside the channel has been dissected and a pocket is formed. A number of different inserts may be positioned within the open pocket including continuous rings, lenses, lenticules, inlays, or any other such insert or implant that may be desirous to provide a necessary correction for defects of the vision or to deliver required medicaments.

Preferably, the initial intrastromal channel is created using an arcuate dissector precisely located and guided with respect to the geometry of the eye. The channel thusly formed establishes a consistent, cleanly dissected periphery accurately located with respect to the center of the cornea. The accurate placement and surgical quality of the dissection at the periphery tends to improve healing and ensures that the dissection does not encroach upon the limbus.

Although such dissectors may be guided manually by the surgeon, a centering guide is preferred. Such guides can be precisely located with respect to the cornea and may be positively held in place mechanically or by vacuum. Exemplar vacuum centering devices can be found, for example, in U.S. Pat. No. 5,403,335, issued Apr. 4, 1995 to Loomis et al. the entirety of which is herein incorporated by reference, copending application Ser. No. 08/796,595 filed on Feb. 7, 1997 titled “IMPROVED DEVICE AND METHOD FOR INSERTING A BIOCOMPATABLE MATERIAL INTO THE CORNEAL STROMA” the entirety of which is herein incorporated by reference, and copending application Ser. No. no. 08/896,754 filed on Jul. 18, 1997 titled “CORNEAL VACUUM CENTERING DEVICE” the entirety of which is herein incorporated by reference.

Referring to FIG. 1, the first step in the general method described above is making an initial incision to allow entry of the various dissecting tools as well as implantation of the appropriate insert. The incision is typically made using a diamond blade located at approximately 1 mm from the limbus. It may be helpful to use a marking tool and vacuum centering guide to mark the location of the incision such as those described in copending application Ser. No. 08/896,792 filed on Jul. 18, 1997 titled “OPTHALMOLOGICAL INSTRUMENTS AND METHODS OF USE” the entirety of which is herein incorporated by reference.

According to the preferred method, Step A of FIG. 1 illustrates a small radial incision 104 about 1-2 mm in length and about 0.2 mm deep below the surface of the cornea 100. The incision is located about 1 mm from the limbus 102. This type of radial incision readily accommodates the insertion of the arcuate dissector tools which will be described in detail with reference to the subsequent method steps.

Other types of incisions may be used. The initial incision may be a circumferential incision about 1 mm from the limbus as shown in FIG. 2. Circumferential incision 105 runs essentially parallel with the outer periphery of the cornea and therefore leaves a larger diameter of cornea free from incision. Since it is desirable that the insert be positioned away from the incision, circumferential incision 105 potentially allows the insert to be positioned at a greater distance from the center of the cornea than that allowed with radial incision 104. The tissue radially interior to circumferential incision 105 may have to be retracted somewhat during subsequent surgical steps to allow for placement and use of subsequent dissecting instruments.

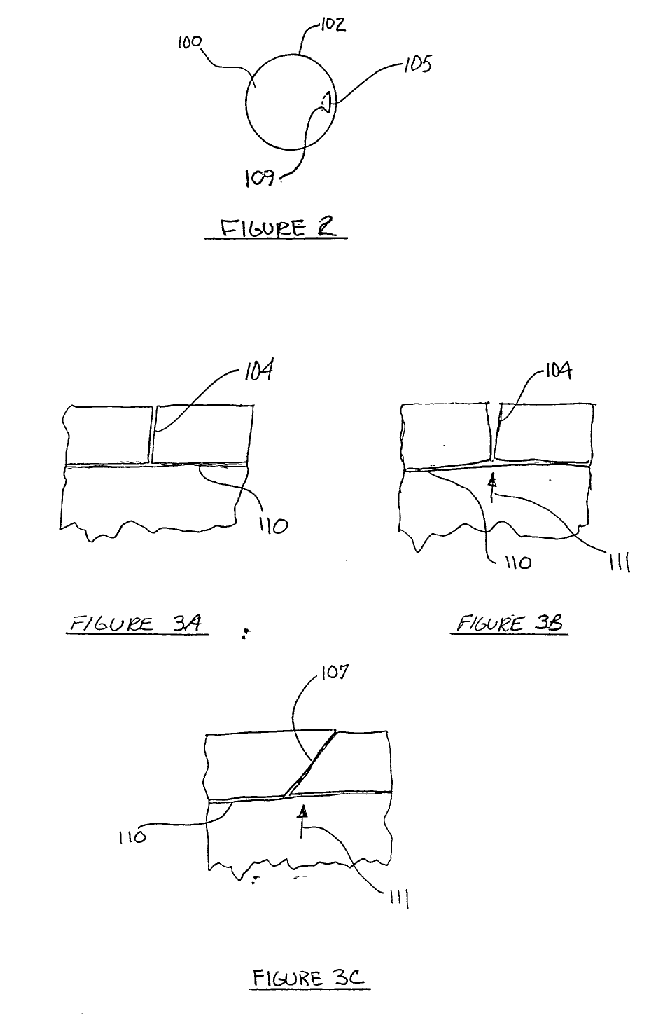

The initial incision, whether radial or circumferential, may be substantially perpendicular to the surface of the cornea or may be at an oblique angle. FIGS. 3A and 3B show a cross-section of the radial incision 104 and subsequently formed intrastromal channel 110 in which an insert is to be placed. Radial incision 104 is roughly perpendicular to the surface of the cornea 100. Referring to FIG. 3B, radial incision 104 is shown deflected under a load in the direction of arrow 111. Such loading, which may be caused when an insert is placed within channel 110, results in vertical radial incision 104 tending to be forced open as shown and thus inhibit optimum healing.

FIG. 3C shows an oblique incision 107 and subsequently formed intrastromal channel 110 in which an insert is to be placed. With this configuration, a load applied in the direction of arrow 111 now results in the tissue on either side of oblique incision 107 being forced into each other. Keeping the tissue in contact even under the stress caused by the placement of an insert within channel 110 promotes healing and lessens scar tissue. Typically the incision is at an angle relative to the surface of the cornea of about 10 degrees to about 80 degrees. More preferably the incision is at an angle of about 30 degrees to 60 degrees.

After the initial incision is made a separation of the lamella beginning at the base of the incision is initiated. This initial separation or pocket facilitates subsequent insertion of a dissecting tool. Initial separation 106 as shown in Step A (or separation 109 as shown in FIG. 2 if a circumferential incision is used) may be accomplished using either or both of a corneal pocketing tool or a stromal spreader. A suitable corneal pocketing tool is disclosed in, co-pending U.S. Patent Application titled “CORNEAL POCKETING TOOL” filed on Dec. 18, 1997, the entirely of which is herein incorporated by reference.

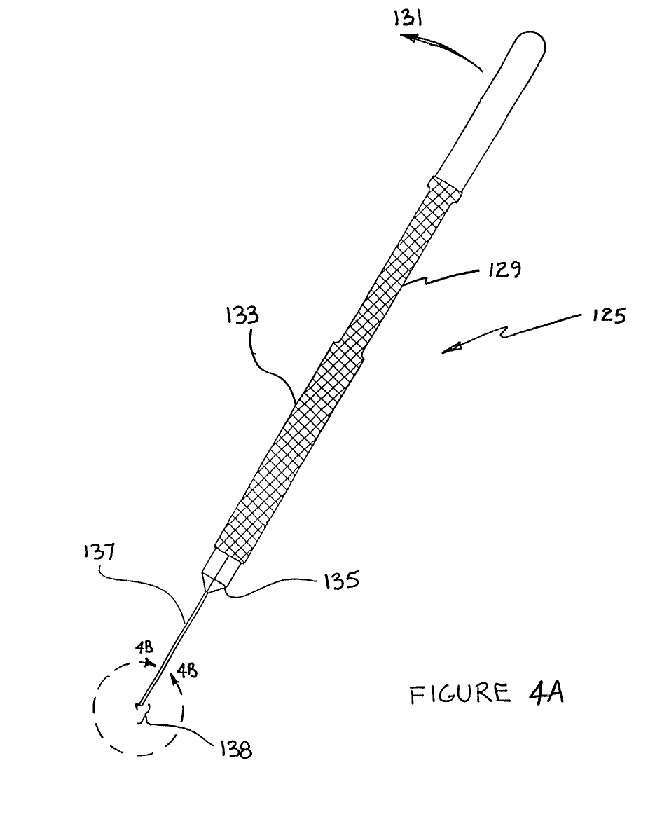

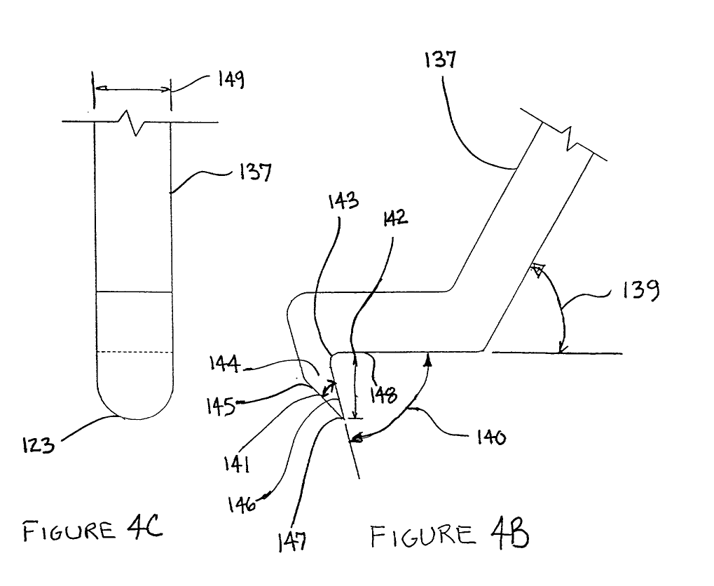

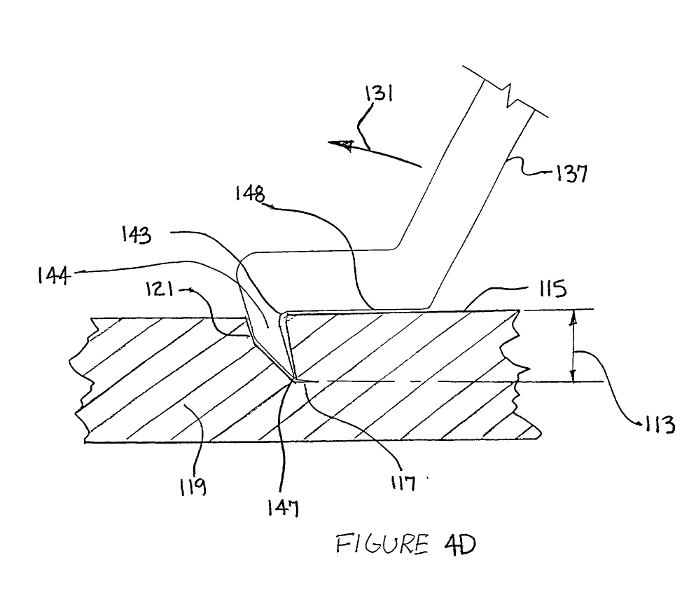

FIG. 4A illustrates a pocketing tool suitable for creating the desired initial separation or pocket. Pocketing tool 125 includes an instrument handle 133 and a thin instrument shaft 137 terminating distally in tip section 138. Instrument handle 133 is typically knurled or coated for purposes of gripping, and may have a flat region 129 to allow the instrument to be marked with any desired identifying data.

The shaft 137 connects to the handle 133 at connecting hub 135. Connecting hub 135 securely attaches the proximal end of the shaft 137 to the handle 133. The entire corneal pocketing tool may be of a single piece of material and ground to the final net shape. Alternatively, shaft 137 may be a separate piece and attached by way of an interference fit with mating features in the hub, or by bonding or welding or the like. The connecting hub 135 may optionally be in the form of a collet or other clamping mechanism that allows substitution of different tip instruments.

The tip section 138 can be seen more clearly in the magnified front and side views illustrated in FIGS. 4B and 4C respectively. Tip section 138 is constructed to have reference region 148, which is adapted to contact the surface of the cornea during use, and is connected proximally to shaft 137 and distally to dissector 144. The reference region 148 may be a generally flat reference surface, may be curved to match the contour of the cornea, or may have any other features or construction which allows the pocketing tool to reference against the surface of the cornea. If the tip section is constructed of wire material, the reference region may be the outside surface of the wire itself.

The shaft 137 is shown disposed at an angle 139 to the plane of the reference region 148. In practice, angle 139 is constructed to provide the surgeon with the optimum manual control and visibility for the particular surgery which is to be performed. Angle 139 is typically between about 10° to 170°, preferably between about 30° to about 90°, most preferably about 60°. A small radius 143 may be provided at the transition between the reference region 148 and the dissector 144. Radius 143 may be from about 0.01 to about 0.05 inches.

The dissector may have a variety of constructions including a relatively thin wire construction or may have a flat profile construction as shown. Dissector 144 has an inner surface 146 and an outer surface 145, and is also disposed in angular relation to reference region 148. The dissector angle 140, shown as the angle between inner surface 146 and reference region 148, may be between about 30° and about 150°, more preferably between about 60° to about 100°, most preferably about 75° as shown.

Inner surface 146 and outer surface 145 generally converge at dissector tip 147. The profile of these converging dissector surfaces are created by grinding, chemical etching, machining, or the like. Dissector tip 147 may be ground sharp, or may be left with a slight radius or even a blunt tip if desired. Grind angle 141 between inner surface 146 and outer surface 145 must provide for enough material in the dissector region to impart the necessary structural rigidity as well as remaining sufficiently thin for insertion into a corneal incision. Grind angle 141 is typically between about 10° to about 50°, more preferably about 25° to about 35°, most preferably about 30°. In the side view illustrated in FIG. 5, dissector 144 may have be formed with a full radius 123 as shown.

To achieve the desired size, structural integrity, and biocompatibility required for proper operation in corneal surgery, pocketing tool 125 is typically made from stainless steel or titanium, preferably anodized titanium. For the purposes of example only, the material thickness in the vicinity of shaft 137 and reference region 148 is typically about 0.014 to 0.020 inches. The side view width 149 of tip section 138 is typically constructed to be somewhat smaller than the width of the incision that will be used, typically less than about one-half of the width of the expected incision. For typical incisions in the range of about 1 mm to about 1.2 mm, width 149 is preferably about 0.02 inches. The downward distance 142 from reference region 148 to dissector tip 147 is preferably constructed to coincide with the desired depth of the corneal pocket to be formed. If, for example, a pocket is to be created at a depth of 0.018 inches from the surface of the cornea, then the instrument will be constructed with a downward distance 142 of about 0.018 inches.

To form a pocket or separation between the stromal layers of the cornea, pocketing tool 125 is inserted into an 121 as shown in FIG. 4D. Incision 121 may be a circumferential type incision a radial type incision as described above. Dissector 144 of corneal pocketing tool 125 is advanced into incision 121 until reference region 148 comes into contact with the surface 115 of the cornea 119. The relatively large contact area of reference region 148 ensures that there will be no significant damage to the corneal tissue as the surgeon applies the downward pressure necessary for insertion of dissector 144 into incision 121. The downward pressure applied to corneal pocketing tool 125 by the surgeon is absorbed by the reference region 148 rather than by dissector tip 147 against the bottom of incision 121. For this reason, the dissecting tip may be relatively sharp to facilitate pocketing, without risk to the surrounding tissue.

Because the distance from reference region 148 to dissector tip 147 corresponds to the desired depth for corneal pocketing (and coincidentally with the bottom of the partial depth incision), once the surgeon appreciates the tactile indication that reference region 148 is in contact with corneal surface 115 it is known that dissector tip 147 is at the proper depth below corneal surface 115. Thus, the surgeon is not required to use the dissecting tip to feel for the bottom of the incision, but instead proper depth of the dissecting tip is indicated by the resistance to further advancement of reference region 148 against corneal surface 115.

With dissector 144 in place within incision 121 as shown in FIG. 4D, an intrastromal separation or pocket is initiated simply by pivoting or rotating the instrument in the direction indicated by arrow 131. This allows dissector 144 and dissecting tip 147 to rotate about radius 143, forcing the stromal layers to delaminate by operation of dissecting tip 147 at the proper depth below corneal surface 115. The amount of rotation required is typically in the range of 10° to 90°, preferably around 45°.

As the instrument is rotated, the depth of the dissecting tip remains controlled in part by reference region 148 as it rotates about radius 143, and a separation or pocket 117 is created. If the width of the incision 121 is greater than the width 149 of the dissector 144, it may be desirable to maneuver dissector 144 across the width of the incision either while holding dissector 144 in the rotated position or by releasing and repositioning dissector (270) to a new position along the width of incision 121.

Once the desired separation or pocket has been started using corneal pocketing tool 125, the various other instruments may then be inserted through the incision to enlarge or otherwise modify the initial pocket. For example, a blunt spatula or spreader may be used to enlarge this initial pocket. Alternatively, a blunt spatula or spreader may be used to make the initial separation. A suitable stromal spreader is described in copending application Ser. No. 08/896,792 filed on Jul. 18, 1997 titled “OPTHALMOLOGICAL INSTRUMENTS AND METHODS OF USE” the entirety of which is herein incorporated by reference.

A spreader instrument 150 is illustrated in FIGS. 5 through 5F. Spreader 150 includes handle 152, extension 154, and tip 156. To provide increased rotational control of spreader 150, a portion of handle 152 is knurled and cutouts 153 are provided in opposing positions for marking the instrument. Extension 154 has a much smaller outside diameter than handle 152, and has a tapering outside diameter that gradually decreases toward the end of extension 154 that joins with tip 156.

Tip 156 is substantially flat and relatively wide and thin as observed in a comparison of FIGS. 5A and 5B. Tip 156 extends from extension 154 at an obtuse angle β to the longitudinal axis of extension 154 and handle 152, as shown in FIG. 5A. The obtuse angle provides the user with a comfortable handle position when tip 156 is inserted into the incision. Tip 156 has a tapering thickness t which decreases in the direction from the extension 154 to tip end 158.

As shown in FIG. 5B, tip end 158 is rounded and is preferably substantially hemispherical. although greater and lesser radii of curvature may be employed to define the tip end. Importantly, the tip end is not knife sharp, but rather, is relatively blunt so as to function to separate tissue along layers, but not to cut. Tip end 158 transitions into tip sides 160 as the curvature of tip end 158 gradually straightens into the substantially straight edges of tip sides 160. Tip sides 160 are sharp, although not knife sharp. A comparison of the relatively dull edge of tip end 158 and the relatively sharp edges of tip sides 160 can be seen by comparing the sectional views of FIGS. 5C and 5D, respectively.

With the arrangement of stromal spreader tip 156 as described, the relatively dull, slightly rounded tip end 158 greatly reduces the risk of perforation of the corneal tissues upon insertion of the tip into the incision. Additionally, by rotating the spreader using handle 152 the stromal layers are can be effectively separated to form a pocket.

FIG. 5E illustrates, in an exaggerated way, the transition between blunt tip end 158 and the relatively sharp edge of tip side 160, which supports the fact that the insertion of the tip presents a relatively low risk of perforation of the stromal tissues. Once the spreader has been inserted, separation can begin through use of sharper side edges 160, together with blunt tip end 158.

FIG. 5F shows a variation of the tip shown in FIG. 5A. In this variation, the joinder of tip 156 and extension 154 is formed at the obtuse angle β to the longitudinal axis of extension 154 and handle 152, the same as shown in FIG. 5A. However, the majority of the tip that is distal to the joinder of the tip and the extension, i.e., tip 156′ is formed at an angle γ with regard to the longitudinal axis of extension 154 and handle 152, and where angle γ is an obtuse angle that is less than obtuse angle β. The remaining features of tip 156′ are essentially the same as those described above with regard to tip 156 in FIGS. 5A-5E.

Referring again to FIG. 1, after creating an initial separation, a blunt, arc-shaped dissector tool 108 is then inserted into the incision 104 and rotated about a central axis to create a circular interlamellar pathway 110 through the stroma, as shown in Step B.

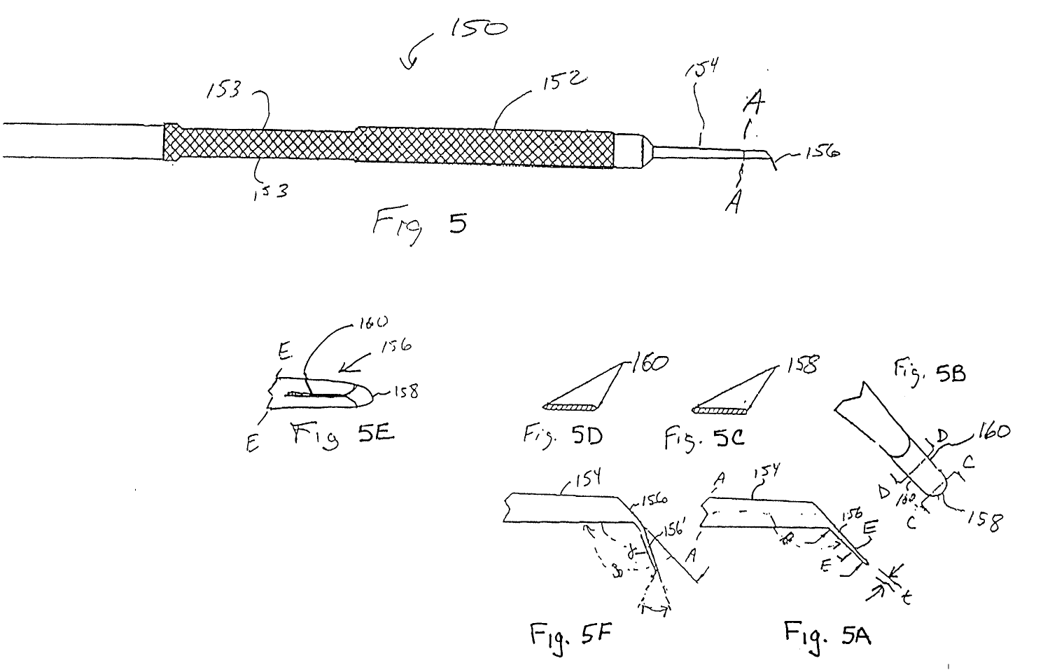

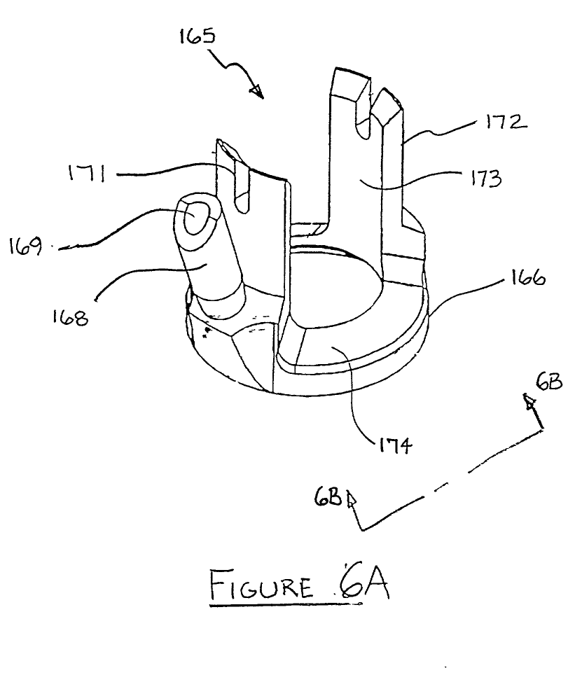

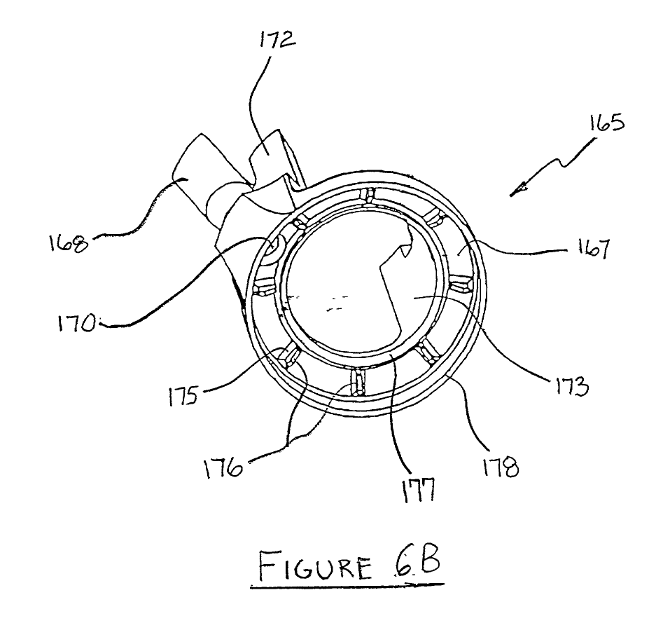

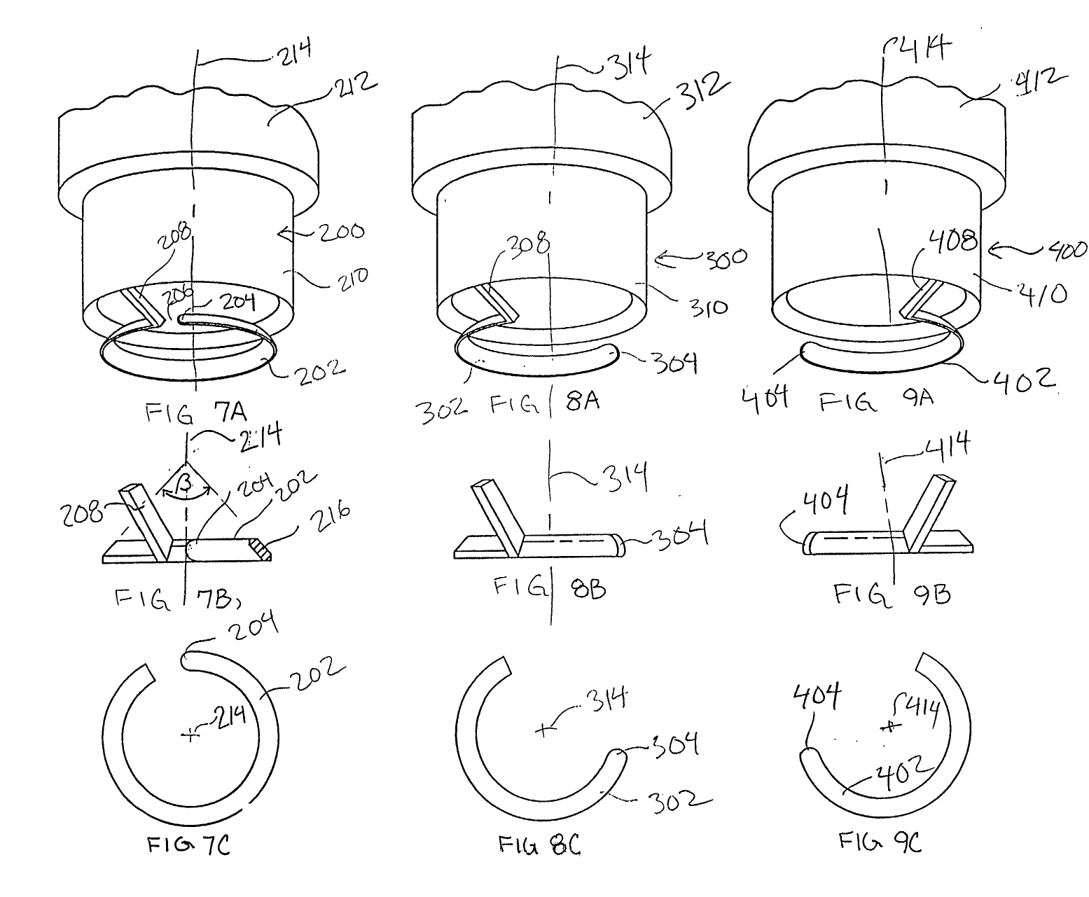

Variations of the dissector tool 108 are shown in FIGS. 7A-7C, 8A-8C, and 9A-9C. As mentioned above, the alignment and rotational movement of the arc-shaped dissector tool may be accomplished either manually by the surgeon or with the aid of a vacuum centering and guiding device. Typically the vacuum centering device is centered over the cornea and fixed to the eye, often by way of a vacuum chamber, to prevent relative motion between the cornea and the vacuum centering device. The vacuum centering device may provide guiding features for various surgical devices such as marking tools used to mark the cornea (i.e., for marking locations of incisions, or circular marks to indicate the inner and outer boundary or a channel to be formed), instruments used to make incisions in the cornea, and the various dissecting instruments.

In this case, where the dissecting tool is to be rotated about a central axis, the vacuum centering device has guiding features which mate with associated features on the dissecting tool to allow controlled rotation with minimal eccentricity and angularity deviation. A detailed description of a vacuum centering guide is given in U.S. Pat. No. 5,403,335 to Loomas et al., in copending application Ser. No. 08/896,754 filed on Jul. 18, 1997 titled “CORNEAL VACUUM CENTERING DEVICE”, both of which having been incorporated by reference above.

A preferred embodiment of a vacuum centering guide for use in the present invention is shown in FIGS. 6A-6C. The vacuum centering guide generally includes a main base portion which includes a sealing chamber and at least one guide support member. Top and Bottom perspective views of vacuum centering device 165 are shown in FIGS. 6A and 6B. Vacuum centering device 165 has a base 166 which includes sealing chamber or vacuum space 167 to which vacuum pressure may be applied by way of tubular connection 168 which has an interior lumen 169 in fluid communication with vacuum port 170 inside the vacuum space 167. Vacuum space 167 is typically bounded by inner annular contacting surface 177 and outer annular contacting surfaces 178 designed to engage the eye and form a vacuum tight seal.

Vacuum centering device 165 has guide support members 171, 172 extending substantially vertically from the base 166 and are generally positioned opposite one another. The guide support members 171, 172 have guide features or surfaces for receiving and accurately positioning a mating surgical instrument. Such guide surfaces may have any suitable shape to mate with the surgical instrument. In the preferred embodiment shown in FIGS. 6A-6C, guide support members 171, 172 have cylindrical guide surfaces 173 for receiving and mating a cylindrical feature on an associated instrument.

Cylindrical guide surfaces such as those just described are particularly useful in the present invention because a number of the dissecting tools described in more detail below require rotation. Cylindrical guide surfaces 173 provide free rotation of a mating cylindrical instrument within vacuum centering device 165 and yet have sufficient height to prevent unacceptable angular movement of the instrument. The maximum angular movement of the surgical instrument allowed by guide surfaces 173 is a function of the clearance between mating surfaces of the surgical instrument and guide surfaces 173, the height of guide surfaces 173, and the total subtended angle of the guide surfaces 173. To improve both visual access and instrument access by the surgeon, guide surfaces 173 subtend less than 360 degrees as shown to allow adequate open area between the guide support members to allow the surgeon to view the eye during surgery as well as access the eye with any necessary surgical instrument.

Preferably, the outer surface 174 of the vacuum centering device is constructed to have a reduced profile, which provides for an improved fit of base 166 between the upper and lower eyelids. Outer surface 174 may be sloped, tapered, flared, chamfered, radiused, or otherwise shaped to provide a lower profile above the surface of the eye. The reduced profile allows vacuum centering device 165 to be fixed to the eye with much less severe retraction of the surrounding eyelids. This in turn provides increased stability of the vacuum centering device as well as increased patient comfort.

A number of radial vanes 175 may be positioned within the vacuum space to provide contact surfaces 176 for contacting the eye when vacuum is applied to the device. These contact surfaces are employed to engage the surface of the eye to provide resistance to rotation of the vacuum centering device 165 against torsional loading, for instance from a rotating surgical instrument such as a dissector. The radial vanes 175 and associated contact surfaces 176 also serve to prevent the surface of the eye to be pulled in too far within vacuum space 167 upon application of vacuum pressure.

Referring now to Steps B and C of FIG. 1, a complete circular interlamellar pathway 110 can be created using a generally arcuate dissector 108. The interlammellar pathway may be created in a single operation using a single circular dissector tool 200 which subtends an arc of approximately 350 degrees, as shown in FIGS. 7A-7C. Alternatively, the circular interlamellar pathway 110 can be completed in two steps using a pair of clockwise 300 and counterclockwise 400 semicircular dissector tools, each subtending an arc of approximately 180-200 degrees, which are shown in FIGS. 8A-8C and 9A-9C. The clockwise 300 and counterclockwise 400 semicircular dissector tools are inserted one at a time into the incision 104 and rotated about a central axis to create two semicircular pathways that join one another at the side of the cornea 100 opposite the incision 104.

An arcuate probe may be inserted into the semicircular pathways to as a check to ensure that the two semicircular pathways meet. If the clockwise and counterclockwise semicircular pathways do not meet exactly, a channel connecting tool similar in construction to the circular dissector tool 200 shown in FIGS. 7A-7C can be used to complete the circular interlamellar pathway 110. This procedure is described in more detail in copending application Ser. No. 08/796,595 filed on Feb. 7, 1997 the entirety of which has been incorporated by reference above.

The completed circular interlamellar pathway 110, as shown in Step C defines the margin or outer boundary of the intracorneal channel that will be formed. Defining the boundary of the intracorneal channel in a controlled and predictable manner this way creates a smooth outer margin which promotes healing. Moreover, it avoids inadvertently extending the intracorneal channel into the limbus 102, which could lead to ingrowth of blood vessels into the cornea 100. Ingrowth of blood vessels from the limbus into the cornea may result in unacceptable impairment of the patient's vision.

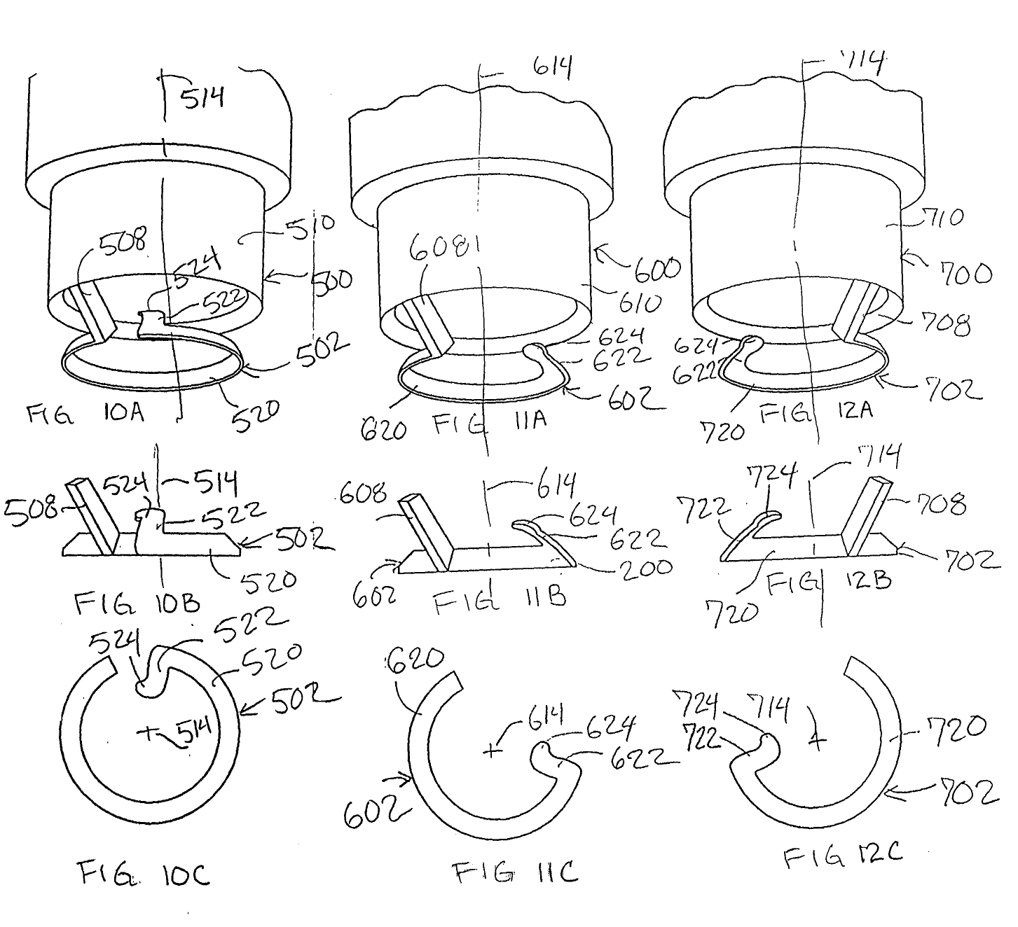

The circular interlamellar pathway 110 is then expanded radially inward in a controlled stepwise fashion to create a wider intracorneal channel 116. This may be accomplished using an arcuate dissector having at least one portion configured to widen the channel as it is advanced into the existing channel formed as described above. Preferably, this is done by introducing a channel-widening dissector tool 112 with a side leg 114 ending in a blunt dissecting tip 118 through the incision 104 and moving the channel-widening dissector tool 112 in an arc-shaped path around the circular interlamellar pathway 110 to widen the intracorneal channel 116, as shown in Step D. As with the initial circular interlamellar pathway 110, the widened intracorneal channel 116 can be created with a single 360 degree circular channel-widening dissector tool 500, as shown in FIGS. 10A-10C, or using a pair of clockwise 600 and counterclockwise 700 180-200 degree semicircular channel-widening dissector tools, shown in FIGS. 11A-11C and 12A-12C. Channel-widening dissector tools with progressively longer side legs 114 are used to expand the channel 116 until the desired width is achieved, as shown in Step E. The side leg 114 and blunt dissecting tip 118 of the channel-widening dissector tool are shaped to conform to the curvature of the anterior surface of the cornea, as shown in the side views in FIGS. 10B, 11B and 12B.

In one aspect of the invention, once the intracorneal channel 116 is widened to the desired width, an intracorneal implant is inserted into the widened channel 116, the implant is positioned within the channel 116 and the incision 104 is closed. In a preferred embodiment the final width of channel 116 allows for the implant to be positioned such that it is radially inward of the incision. In the case of a 1 mm radial incision, the final width of the channel would be slightly more than the width of the implant plus 1 mm. Intracorneal implants which benefit from this technique include, but are not limited to, a split ring intracorneal implant 120, as shown in Step F, or a segmented ring intracorneal implant 122, as shown in Step G. The widening of the intracorneal channel 116 allows the intracorneal implant 120, 122 to be positioned remotely from the incision 104, as shown, so that no unnecessary stress is exerted on the incision 104 during healing.

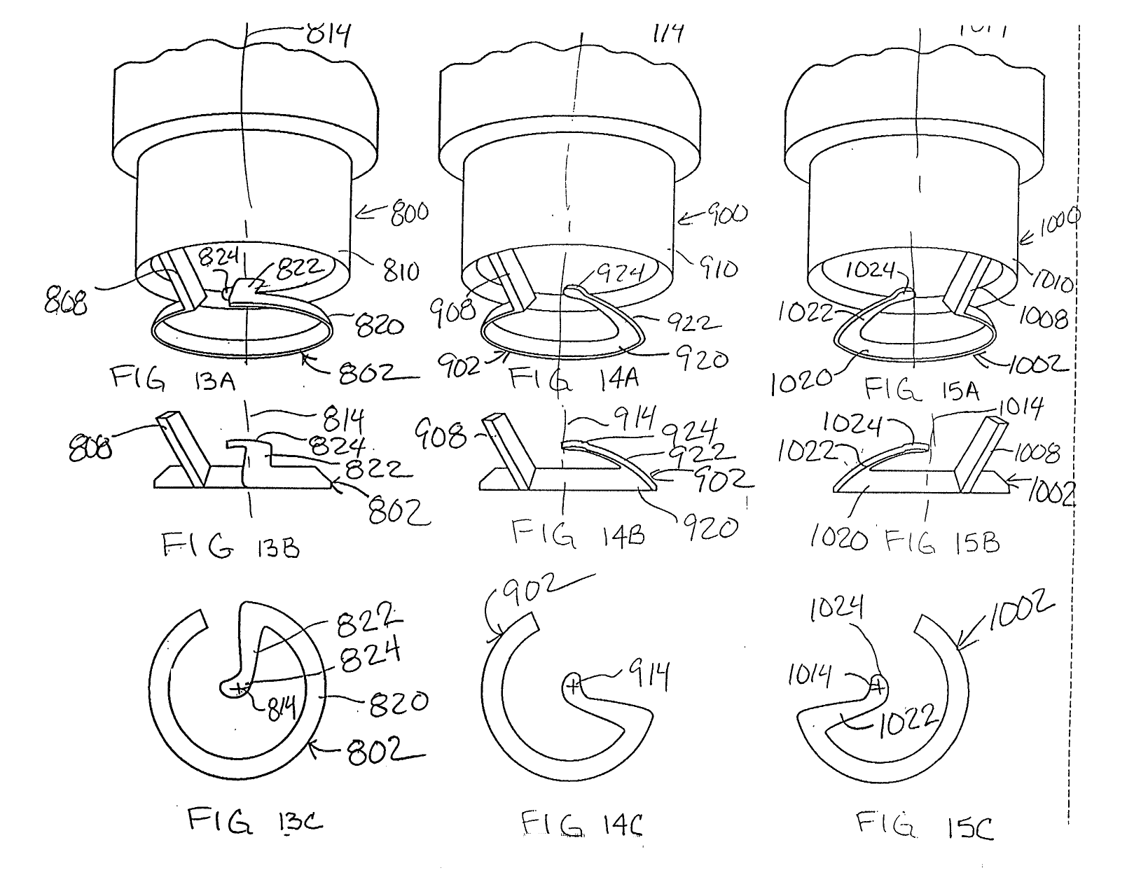

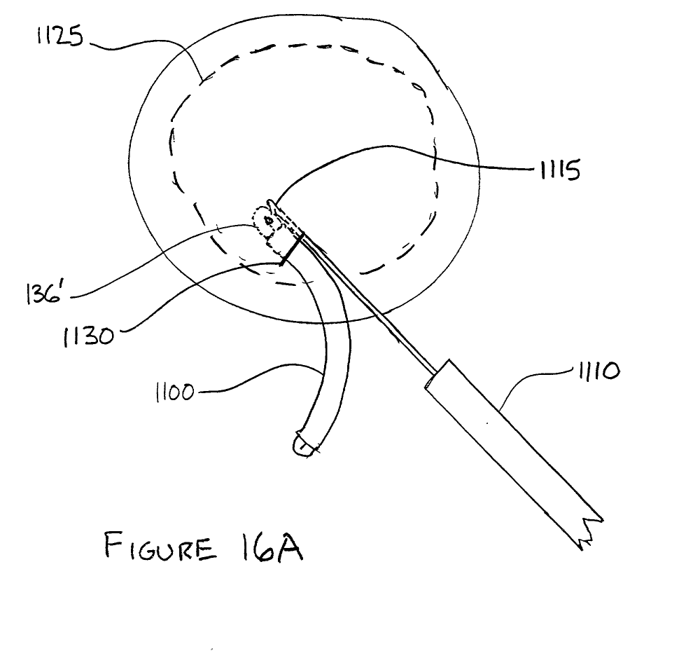

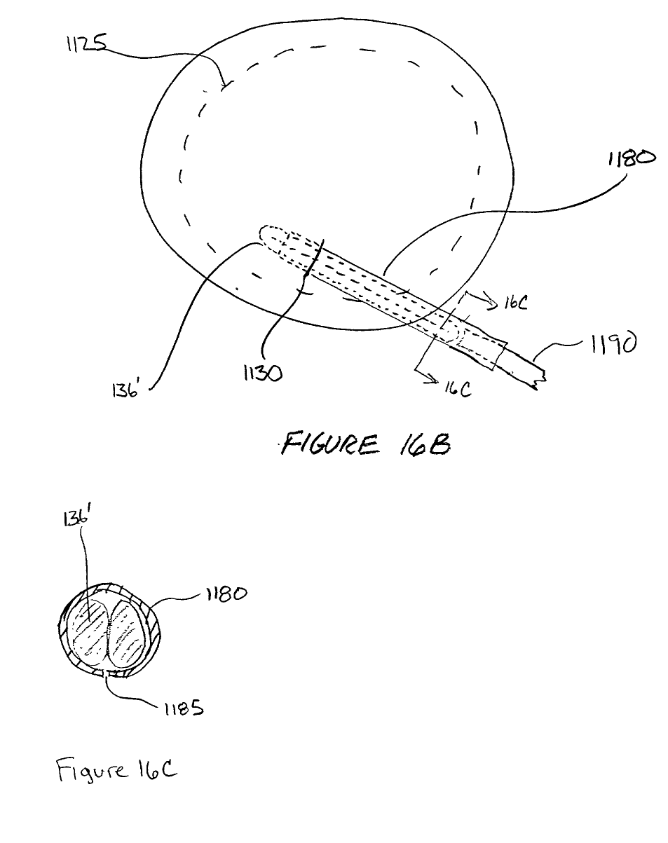

In another aspect of the invention, the intracorneal channel 116 is widened to the point that it creates an intracorneal pocket 124. In a preferred embodiment of the surgical method, a pocket-forming dissector tool 126 with a side leg 128 that is slightly longer than the radius of the initial circular interlamellar pathway 110 is inserted through the incision 104 into the widened intracorneal channel 116 of Step E and the pocket-forming dissector tool 126 is rotated about a central axis, as shown in Step H, to create an intracorneal pocket 124, which is shown completed in Step I. The intracorneal pocket 124 can be created with a single 360 degree dissector tool 800, as shown in FIGS. 13A-13C, or using a pair of clockwise 900 and counterclockwise 1000 180-200 degree semicircular dissector tools, shown in FIGS. 14A-14C and 15A-15C. The blunt dissecting tip 130 and the side leg 128 of the pocket-forming dissector tool 126 are shaped to conform to the curvature of the anterior surface of the cornea, as shown in the side views in FIGS. 8B, 9B and 10B.

In an alternate embodiment of the surgical method, the intracorneal pocket 124 can be completed by inserting a curved, blunt dissecting spatula 132 or similar probe through the incision 104 and dissecting the lamellae across the optical zone 134 of the cornea 100, as shown in Step O. Because the outer boundary of the intracorneal pocket 124 has already been carefully and precisely defined by the circular interlamellar pathway 110 in Step C, there is less concern about creating irregular edges at the margin of the pocket with the dissecting spatula 132 or of overshooting and dissecting the cornea 100 into the limbus 102 which could cause healing problems for the cornea 100.

It should also be noted that the intracorneal pocket forming steps shown in FIGS. 1, Step O or Step H could alternatively be performed starting from the initial circular interlamellar pathway 110 in Step C without performing the intermediate channel widening step of Step D. However, a controlled, stepwise inward expansion of the intracorneal channel 116 is preferred for achieving the best results when creating an intracorneal pocket 124. In addition, the initial channel 110 may be formed radially inward from the position shown and widened outwardly using a dissector having a leg extending radially outward. The pocket could then be completed by dissecting radially inward from the initial channel.

Once the intracorneal pocket 124 is completed, as shown in Step 1, an intracorneal implant is inserted into the pocket 124. Intracorneal implants appropriate to this technique include a continuous ring intracorneal implant 136, as shown in Steps J, K and L, an intracorneal lens or lenticule implant 138, as shown in Steps M and N, or any other appropriate inlay or dye treatment used to correct defects of the vision.

Step J shows a continuous ring intracorneal implant 136′, which has been folded in half, being inserted through the incision 104 into the completed intracorneal pocket 124. Once the folded continuous ring intracorneal implant 136′ is fully inserted into the intracorneal pocket 124, the continuous ring intracorneal implant 136 is unfolded and positioned around the optical zone 134 of the cornea 100, as shown in Step K.

Folded continuous ring intracorneal implant 136′ may be inserted in a number of ways. A standard pair of forceps may be used to grip the implant a small distance away from the incision and advance the implant towards the incision and into the pocket in a series of small increments.

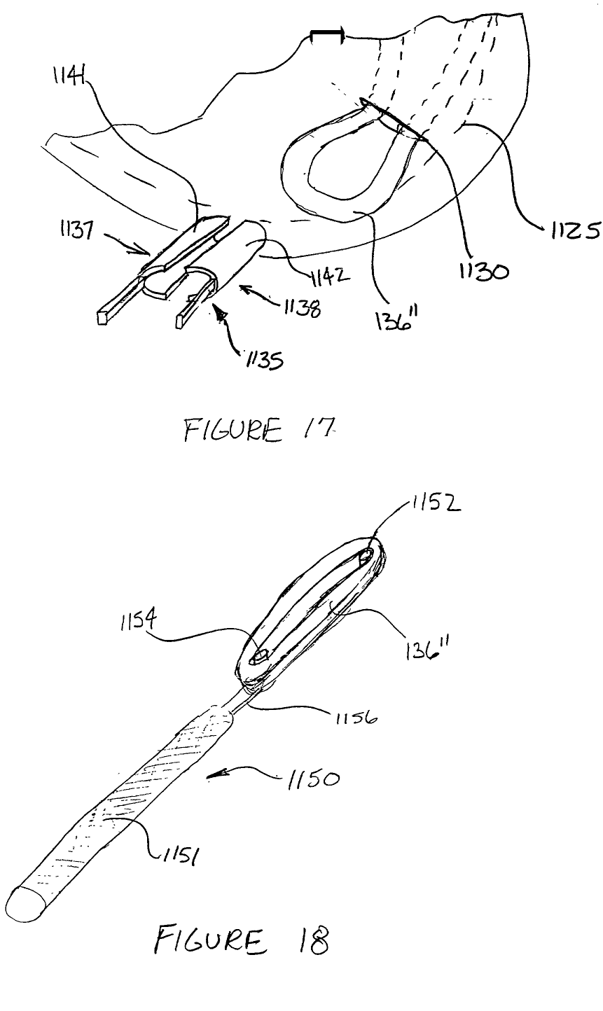

Another technique of introducing the implant into a pocket is shown in FIG. 16A. The continuous ring intracorneal implant 136′ is inserted into pocket 1125 by assembling the implant into a tube element 1100 leaving an end portion of the implant extending from the tube, inserting at least a portion of the tube containing the implant into the pocket, and then using a hook instrument 1110 to pull the ring from the tube.

Preferably tube 1100 is arc-shaped having a radius that is approximately the same as that of the continuous ring intracorneal implant 136′ in the unfolded state. The tube 1100 is inserted a distance into the pocket 1125 through an incision such as radial incision 1130. Hook instrument 1110 is inserted through incision 1130 and manipulated to engage the portion of the implant extending from the tube. Hook instrument 1110 is then advanced towards the incision to pull the implant from the tube 1100. Tube 1100 is then withdrawn from the cornea. Alternatively, the ring implant may be pulled from the tube by way of an additional incision (not shown) on the opposite side of the cornea.

Referring now to FIG. 16B, a straight tube 1180 may also be employed to insert the continuous ring intracorneal implant 136′ into the intracorneal pocket 124. Preferably, at least a portion of straight tube 1180 is split to allow for assembly of the implant into the tube. As seen in FIG. 16C, the tube may have a longitudinal split 1185. It may be desirable to provide the implant preinstalled into the tube in a presterilized assembly or kit. Also with reference to FIG. 16B, a pushing device, such as plunger 1190, may be used to deploy the continuous ring intracorneal implant 136′ into the pocket. The pusher or plunger typically works in cooperation with the straight tube 1180 or curved tube 1100 allow the implant to be ejected into the pocket.

The continuous ring implant does not have to be folded for insertion. A continuous ring intracorneal implant 136″ which is made of a flexible material may be stuffed through the incision without folding, as shown in Step L. Once the continuous ring intracorneal implant 136″ is fully inserted into the intracorneal pocket 124, the continuous ring intracorneal implant 136 is straightened out and positioned around the optical zone 134 of the cornea 100, as shown in Step K. Preferably, the continuous ring intracorneal implant 136 is positioned remotely from the incision 104, as shown, so that no unnecessary stress is exerted on the incision 104 during healing.

The implant 136″ may be implanted by a variety of techniques. Standard forceps may be used to grip and advance the implant into the pocket through the incision as described above with reference to Step J above. Further, the forceps may be provided with specialized end effectors 1137, 1138 to more positively grip and insert implant 136″. End effector 1135 is shown having two opposing concave clamp elements 1141, 1142 for gripping implant 136″ when moved respectively in the directions of arrow 1137 and arrow 1138. When closed, the clamp elements 1141, 1142 form a smooth outer profile that may be inserted through incision 1130 and into pocket 1125. Implant 136″ may be gripped by clamp elements 1141, 1142 and then that part of the implant captured by the clamp elements 1141, 1142 may be guided through the incision before release.

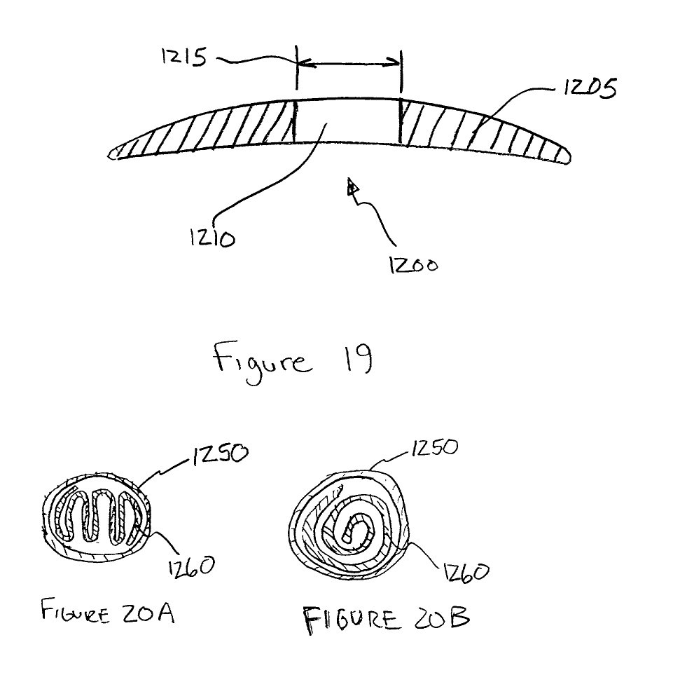

The implant 136″ may also be inserted into the pocket in a stretched state. The implant may be assembled over features adapted to maintain the implant in a stretched state. Once inserted into the corneal pocket, the insert is released from the stretched state and then positioned within the pocket as described above. In the embodiment of FIG. 18, insertion tool 1150 includes a handle 1151 and a thin support member 1156. Support member 1156 has a distal protrusion 1152 and a proximal protrusion 1154. The protrusions may be of a great number of shapes adapted to maintain the position of implant 136″ in a stretched state. As shown in FIG. 18, protrusions 1152 and 1154 are shown generally as cylindrical pegs. In operation, implant 136″ is positioned or stretched over distal protrusion 1152 and proximal protrusion 1154. By manipulation of handle 1151, an implant 136″ is inserted into the pocket through any of the types of incisions described above and then released from the protrusions.

It may be desirable to insert implant 136″ only until the proximal protrusion 1154 is just outside of the incision. At that point, the implant may be easily released from the proximal protrusion and because of its elastic properties will pull completely into the incision as the implant springs towards the distal protrusion 1152 within the pocket. The implant may then be released from the distal protrusion 1152 by manually manipulation of the insertion tool 1150 and positioned as shown in Step K.

The continuous ring material may be made from any material that is sufficiently flexible to be folded or stretched as required without sustaining substantial permanent deformation. Suitable biocompatible continuous ring materials for the methods described above include polyurethanes, elastomers, polyvinyl alcohols (PVAs), poly vinyl pyrolidone (PVPs), block copolymers, and hydrogels. Preferably the material is a soft biocompatible polymer such as silicone or implantable acrylic hydrogels.

The size of the continuous ring is limited by the size of the incision but are typically about 0.2 to 1.5 mm wide and about 0.1 to 1.0 mm thick. The cross-section of the rings may be of any shape suitable to effect the desired correction of vision defects including round, ovaloid, non-ovaloid and polygonal shapes. Preferably, the continuous ring has a hexagonal shape. Step M shows an intracorneal lens implant 138′ (which has been folded in half) being inserted through the incision 104 and into the completed intracorneal pocket 124. Once the folded intracorneal lens implant 138′ is fully inserted into the intracorneal pocket 124, the intracorneal lens implant 138 is unfolded and positioned within the optical zone 134 of the cornea 100, as shown in Step N. Preferably, the intracorneal lens implant 138 is positioned remotely from the incision 104, as shown, so that no unnecessary stress is exerted on the incision 104 during healing. The incision 104 is then closed and the cornea is allowed to heal.

There are many types of implants and methods of insertion that are applicable for insertion into intracorneal pocket 124. The lens or lenticule may be adapted to have an optical power for the correction of vision or may be constructed to effect a desired change in the radius of curvature of the cornea. The lens or lenticule may be one-piece or have a number of sections of varying properties. The lens may be designed to increase the normal depth of focus of the eye either by means of a small aperture or having a lens body with predetermined areas of different refractive or opacity characteristics.

Referring to the illustrative multi-section lens embodiment shown in FIG. 19, lens 1200 is generally disc shaped and has an inner portion 1210 having a diameter 1215 and outer portion 1205. The inner portion 1210 may be configured from either relatively hard or soft biocompatible material suitable for intracorneal lens construction. The inner portion 1210 is preferably constructed of optically clear material or in the alternative may comprise a through hole. The power of the optically clear inner portion 1210 can be varied to correct for myopia, hyperopia, or astigmatism if desired. The diameter 1215 of inner portion 1210 is typically from about 0.50 mm to 2.00 mm. The outer portion 1205, is preferably constructed to allow glucose and other nutrients to flow through it. For example, the outer portion 1205 may be constructed of hydrogel.

Like the continuous ring, the lenses, lenticules, and inlays may be inserted into an intracorneal pocket in a number of ways. They may be folded in any convenient manner and manipulated through the incision and into the pocket using standard forceps, or forceps with specialized end effectors as described above.

Such implants may also be folded up within an introducer tube or barrel having a sufficiently low-profile to allow delivery of the implant through a minimal incision. A lens, lenticule, or inlay type implant may be folded in any suitable manner which allows them to be delivered into the pocket through a small incision, and then unfolded and positioned within the pocket. Within the introducer barrel, the implant may be folded or rolled or both, or may have a more random configuration if the implant is forced into the barrel in an uncontrolled manner (e.g. by fluid pressure). FIG. 20A shows an implant 1260 folded in an alternating fan-fold arrangement within introducer barrel 1250. FIG. 20B shows implant 1260 rolled up within introducer barrel 1250.

The implant 1250 may be placed in the introducer barrel in a number of ways. The introducer barrel may include a section having a longitudinal split or opening (as discussed with reference to FIG. 16C above) to allow the folded implant to be installed within the introducer barrel. The proximal end of the barrel may optionally include a funnel-type transition structure that facilitates insertion of the implant through the end of the introducer barrel. The introducer barrel may also have a proximal chamber portion (not shown) which accepts an unfolded implant and includes a source of fluid pressure to force the implant from the chamber into the introducer barrel. As discussed above with reference to the continuous ring, the implant may be deployed from the introducer barrel by use of a surgical instrument, for instance having a hook, or by use of a plunger to push the implant, or by use of fluid pressure (e.g. a syringe or the like).

In all of the variations of the method described above, the small size of the initial incision 104, the isolation of the incision 104 from any stress due to the presence of the implant, and the precise and controlled boundary of the channel or pocket will reduce the likelihood of scar tissue formation, which will contribute to the positive results of the vision correction surgery. In addition, in the unlikely occurrence that the results of the vision correction surgery are unsatisfactory or if there are other complications, the reduced scar tissue formation will contribute to the reversibility of the procedure. Clinical experience with split ring intracorneal implants 120, similar the one shown in FIG. 1, Step F, has shown that within approximately eight weeks after surgical removal of the split ring intracorneal implant 120, the patient's vision will substantially return to its previous level before implantation.

Variations of the dissector tool 108 for creating a circular interlamellar pathway 110 through the corneal stroma, shown generically in FIG. 1, Step B, are shown in FIGS. 7A-7C, 8A-8C, and 9A-9C. Referring to these figures, the dissector tool for creating the interlamellar dissection will be described in more detail below.

FIG. 7A is a perspective view of the distal end of a circular dissector tool 200 for creating the circular interlamellar pathway 110 through the corneal stroma in a single step. The circular dissector tool 200 has a circular dissector blade 202 which subtends an arc approaching as close as practically possible to a full circle. Typically, the circular dissector blade 202 will subtend an arc of approximately 350 degrees, leaving a small gap 206 which facilitates insertion of the blunt, dissecting tip 204 of the circular dissector blade 202 through the incision 104 in the cornea 100. The circular dissector blade 202 is attached to the barrel 210 of the circular dissector tool 200 by a support arm 208. The circular dissector blade 202 may extend clockwise or counterclockwise from the support arm 208. In one particularly preferred embodiment of the circular dissector tool 200, the length and diameter of the barrel 210 are chosen so that the circular dissector tool 200 can operate in cooperation with the vacuum centering guide described above. Alternatively the proximal end 212 of the circular dissector tool 200 can be extended to provide a handle for a manually operated version of the tool.

The circular dissector blade 202 of the circular dissector tool 200 is shown in a partially cut-away side view in FIG. 7B and in a distal end view in FIG. 7C. The arc of the circular dissector blade 202 is centered around and in a plane perpendicular to the central axis of rotation 214 of the circular dissector tool 200. In FIG. 7B, the circular dissector blade 202 is partially cut-away to show the cross section 216 of the blade. The cross section 216 of the circular dissector blade 202 is preferably hexagonal with two of the parallel sides longer than the remaining four, as shown. Alternatively, the circular dissector blade 202 may have a rectangular, oval or oblong cross section. The circular dissector blade 202 is configured so that the longer parallel sides form a cone angle β having a vertex which is coincident with the central axis of rotation 214 of the circular dissector tool 200. The cone angle β has a value of approximately 112 degrees (+/−30 degrees) which permits the circular dissector blade 202 to create a circular interlamellar pathway 110 through the cornea 100 which is approximately parallel to the anterior surface of the cornea and to the internal lamellae of the corneal stroma.

FIGS. 8A-8C and FIGS. 9A-9C illustrate a pair of clockwise 300 and counterclockwise 400 semicircular dissector tools which can be used sequentially to complete the circular interlamellar pathway 110 in two steps. FIG. 8A is a perspective view of the distal end of the clockwise semicircular dissector tool 300. The clockwise semicircular dissector tool 300 has a clockwise semicircular dissector blade 302 which subtends an arc of approximately 180-200 degrees and ends in a blunt, dissecting tip 304. FIG. 8B is a side view of the clockwise semicircular dissector blade 302 and FIG. 8C is a distal end view of the clockwise semicircular dissector blade 302. The clockwise semicircular dissector blade 302 extends clockwise from the support arm 308 which attaches it to the barrel 310 of the clockwise semicircular dissector tool 300 when viewed from the proximal end 312 of the tool 300. The arc of the clockwise semicircular dissector blade 302 is centered around and in a plane perpendicular to the central axis of rotation 314 of the tool 300. The clockwise semicircular dissector blade 302 is configured to have a cone angle of approximately 112 degrees (+/−30 degrees) which permits the blade 302 to create a semicircular interlamellar pathway 110 through the cornea 100 which is approximately parallel to the anterior surface of the cornea and to the internal lamellae of the corneal stroma.

The counterclockwise semicircular dissector tool 400 is a mirror image of the clockwise semicircular dissector tool 300. FIG. 9A is a perspective view of the distal end of the counterclockwise semicircular dissector tool 400. The counterclockwise semicircular dissector tool 400 has a counterclockwise semicircular dissector blade 402 which subtends an arc of approximately 180-200 degrees and ends in a blunt, dissecting tip 404. FIG. 9B is a side view of the counterclockwise semicircular dissector blade 402 and FIG. 9C is a distal end view of the counterclockwise semicircular dissector blade 402. The counterclockwise semicircular dissector blade 402 extends counterclockwise from the support arm 408 which attaches it to the barrel 410 of the counterclockwise semicircular dissector tool 400 when viewed from the proximal end 412 of the tool 400. The arc of the counterclockwise semicircular dissector blade 402 is centered around and in a plane perpendicular to the central axis of rotation 414 of the tool 400. The counterclockwise semicircular dissector blade 402 is configured to have a cone angle which is approximately parallel to the anterior surface of the cornea and to the internal lamellae of the corneal stroma. Depending upon location of the cornea, the cone angle is preferably approximately 112 degrees (+/−30 degrees) which permits the blade 402 to create a semicircular interlamellar pathway 110 through the cornea 100. The clockwise 300 and counterclockwise 400 semicircular dissector tools may be configured to operate with the vacuum centering guide described above or they may be configured for manual operation.

Variations of the channel-widening dissector tool 112 for expanding the interlamellar pathway 110 to create a wider intracorneal channel 116, shown generically in FIG. 1, Step D, are shown in FIGS. 10A-10C, 11A-11C, and 12A-12C. Referring to these figures, the dissector tool for expanding the interlamellar pathway will be described in more detail below.

FIG. 12A is a perspective view of the distal end of a circular channel-widening dissector tool 500 for expanding the interlamellar pathway 110 to create a wider intracorneal channel 116 in a single step. The circular channel-widening dissector tool 500 has a channel-widening dissector blade 502 with an approximately circular segment 520, subtending an arc of approximately 350 degrees, attached to the barrel 510 of the circular channel-widening dissector tool 500 by a support arm 508. The circular channel-widening dissector blade 502 may extend clockwise or counterclockwise from the support arm 508. A side leg 522 extends radially inward from the distal end of the circular segment 520, terminating in a blunt dissecting tip 524. The entire channel-widening dissector blade 502, including the blunt dissecting tip 524, subtends an arc of approximately 360 degrees. The circular channel-widening dissector tool 500 may be configured to operate with the vacuum centering guide described above or it may be configured for manual operation.

The channel-widening dissector blade 502 of the circular channel-widening dissector tool 500 is shown in a side view in FIG. 10B and in a distal end view in FIG. 10C. The arc of the circular segment 520 is centered around and in a plane perpendicular to the central axis of rotation 514 of the circular channel-widening dissector tool 500. The radially extending side leg 522 extends upward from the plane of the circular segment 520. Channel-widening dissector tools 500 with progressively longer side legs 522 are used to expand the channel 116 in a stepwise fashion until the desired width is achieved. The circular segment 520, the side leg 522 and the blunt dissecting tip 524 of the channel-widening dissector tool 500 are shaped to conform to the curvature of the anterior surface of the cornea so that the widened intracorneal channel 116 will remain approximately parallel to internal lamellae of the corneal stroma. This may involve having a side leg configured with multiple radii of curvature or a radially variable radius of curvature so that it matches the geometry of the cornea that is to be dissected. The geometry of the channel-widening dissector blade 502 can most easily be envisioned by picturing it as though the entire channel-widening dissector blade 502 is cut out of the side of a sphere with a radius just slightly smaller than the radius of curvature of the cornea 100.

FIGS. 11A-11C and FIGS. 12A-12C illustrate a pair of clockwise 600 and counterclockwise 700 semicircular channel-widening dissector tools which can be used to widen the intracorneal channel 116 in two or more sequential steps. FIG. 11A is a perspective view of the distal end of the clockwise semicircular channel-widening dissector tool 600. The clockwise semicircular channel-widening dissector tool 600 has a clockwise semicircular channel-widening dissector blade 602 with an approximately 180-200 degree semicircular segment 620 attached to the barrel 610 of the semicircular channel-widening dissector tool 600 by a support arm 608. A side leg 622 extends radially inward from the distal end of the semicircular segment 620, terminating in a blunt dissecting tip 624. FIG. 11B is a side view of the clockwise semicircular channel-widening dissector blade 602 and FIG. 11C is a distal end view of the clockwise semicircular channel-widening dissector blade 602. The arc of the clockwise semicircular channel-widening dissector blade 602 is centered around and in a plane perpendicular to the central axis of rotation 614 of the tool 600. The radially extending side leg 622 extends upward from the plane of the circular segment 620. Channel-widening dissector tools 600 with progressively longer side legs 622 are used to expand the channel 116 in a stepwise fashion until the desired width is achieved. The circular segment 620, the side leg 622 and the blunt dissecting tip 624 of the channel-widening dissector tool 600 are shaped to conform to the curvature of the anterior surface of the cornea.

The counterclockwise semicircular channel-widening dissector tool 700 is a mirror image of the clockwise semicircular channel-widening dissector tool 600. FIG. 12A is a perspective view of the distal end of the counterclockwise semicircular channel-widening dissector tool 700. The counterclockwise semicircular channel-widening dissector tool 700 has a counterclockwise semicircular channel-widening dissector blade 702 with an approximately 180-200 degree semicircular segment 720 attached to the barrel 710 of the semicircular channel-widening dissector tool 700 by a support arm 708. A side leg 722 extends radially inward from the distal end of the semicircular segment 720, terminating in a blunt dissecting tip 724. FIG. 12B is a side view of the counterclockwise semicircular channel-widening dissector blade 702 and FIG. 12C is a distal end view of the counterclockwise semicircular channel-widening dissector blade 702. The arc of the counterclockwise semicircular channel-widening dissector blade 702 is centered around and in a plane perpendicular to the central axis of rotation 714 of the tool 700. The radially extending side leg 722 extends upward from the plane of the circular segment 720. Channel-widening dissector tools 700 with progressively longer side legs 722 are used to expand the channel 116 in a stepwise fashion until the desired width is achieved. The circular segment 720, the side leg 722 and the blunt dissecting tip 724 of the channel-widening dissector tool 700 are shaped to conform to the curvature of the anterior surface of the cornea. The clockwise 600 and counterclockwise 700 semicircular channel-widening dissector tools may be configured to operate with the vacuum centering guide described above or they may be configured for manual operation.

Variations of the pocket-forming dissector tool 126 for expanding the intracorneal channel 116 into an intracorneal pocket 124, shown generically in FIG. 1, Step H, are shown in FIGS. 13A-13C, 14A-14C, and 15A-15C. Referring to these figures, the dissector tool for creating the interlamellar dissection will be described in more detail below.

FIG. 13A is a perspective view of the distal end of a circular pocket-forming dissector tool 800 for expanding the intracorneal channel 116 into an intracorneal pocket 124 in a single step. The circular pocket-forming dissector tool 800 has a pocket-forming dissector blade 802 with an approximately circular segment 820, subtending an arc of approximately 350 degrees, attached to the barrel 810 of the circular pocket-forming dissector tool 800 by a support arm 808. The circular pocket-forming dissector blade 802 may extend clockwise or counterclockwise from the support arm 808. A side leg 822, which is slightly longer than the radius of the initial circular interlamellar pathway 110, extends radially inward from the distal end of the circular segment 820, terminating in a blunt dissecting tip 824. The entire pocket-forming dissector blade 802, including the blunt dissecting tip 824, subtends an arc of approximately 360 degrees. The circular pocket-forming dissector tool 800 may be configured to operate with the vacuum centering guide as described above or it may be configured for manual operation.

The pocket-forming dissector blade 802 of the circular pocket-forming dissector tool 800 is shown in a side view in FIG. 13B and in a distal end view in FIG. 13C. The arc of the circular segment 820 is centered around and in a plane perpendicular to the central axis of rotation 814 of the circular pocket-forming dissector tool 800. The radially extending side leg 822 extends upward from the plane of the circular segment 820. The circular segment 820, the side leg 822 and the blunt dissecting tip 824 of the pocket-forming dissector tool 800 are shaped to conform to the curvature of the anterior surface of the cornea so that the intracorneal pocket 124 formed will remain approximately parallel to internal lamellae of the corneal stroma.

FIGS. 14A-14C and FIGS. 15A-15C illustrate a pair of clockwise 900 and counterclockwise 1000 semicircular pocket-forming dissector tools which can be used to widen the intracorneal channel 116 into an intracorneal pocket 124 in two sequential steps. FIG. 14A is a perspective view of the distal end of the clockwise semicircular pocket-forming dissector tool 900. The clockwise semicircular pocket-forming dissector tool 900 has a clockwise semicircular pocket-forming dissector blade 902 with an approximately 180-200 degree semicircular segment 920 attached to the barrel 910 of the semicircular pocket-forming dissector tool 900 by a support arm 908. A side leg 922, which is slightly longer than the radius of the initial circular interlamellar pathway 110, extends radially inward from the distal end of the semicircular segment 920, terminating in a blunt dissecting tip 924. FIG. 14B is a side view of the clockwise semicircular pocket-forming dissector blade 902 and FIG. 14C is a distal end view of the clockwise semicircular pocket-forming dissector blade 902. The arc of the clockwise semicircular pocket-forming dissector blade 902 is centered around and in a plane perpendicular to the central axis of rotation 914 of the tool 900. The radially extending side leg 922 extends upward from the plane' of the circular segment 920. The circular segment 920, the side leg 922 and the blunt dissecting tip 924 of the pocket-forming dissector tool 900 are shaped to conform to the curvature of the anterior surface of the cornea.

The counterclockwise semicircular pocket-forming dissector tool 1000 is a mirror image of the clockwise semicircular pocket-forming dissector tool 900. FIG. 15A is a perspective view of the distal end of the counterclockwise semicircular pocket-forming dissector tool 1000. The counterclockwise semicircular pocket-forming dissector tool 1000 has a counterclockwise semicircular pocket-forming dissector blade 1002 with an approximately 180-200 degree semicircular segment 1020 attached to the barrel 1010 of the semicircular pocket-forming dissector tool 1000 by a support arm 1008. A side leg 1022, which is slightly longer than the radius of the initial circular interlamellar pathway 110, extends radially inward from the distal end of the semicircular segment 1020, terminating in a blunt dissecting tip 1024. FIG. 15B is a side view of the counterclockwise semicircular pocket-forming dissector blade 1002 and FIG. 15C is a distal end view of the counterclockwise semicircular pocket-forming dissector blade 1002. The arc of the counterclockwise semicircular pocket-forming dissector blade 1002 is centered around and in a plane perpendicular to the central axis of rotation 1014 of the tool 1000. The radially extending side leg 1022 extends upward from the plane of the circular segment 1020. The circular segment 1020, the side leg 1022 and the blunt dissecting tip 1024 of the pocket-forming dissector tool 1000 are shaped to conform to the curvature of the anterior surface of the cornea. Again, this may involve a construction having multiple or varying cone angles. The clockwise 900 and counterclockwise 1000 semicircular pocket-forming dissector tools may be configured to operate with the vacuum centering guide as described above or they may be configured for manual operation.

The dissecting tools described above, when connected to a barrel for guiding within a vacuum centering guide as shown and described above, allow the surgeon visual access to the entire procedure by virtue of the fact that the arcuate dissectors are smaller than the diameter of the barrel.

This invention has been described and exemplified in some detail. Those having ordinary skill in this art would recognize variations and equivalents that would be well within the scope of the invention disclosed here but perhaps outside the scope of the appended claims. It is applicants intention that these equivalent variations be included within the scope of this invention.

What is claimed is:

1. A method for correcting defects in vision comprising the steps of:
a) cutting a small incision in the anterior surface of the cornea of an eye;
b) creating a circular intracorneal channel originating at said incision;
c) widening said circular intracorneal channel to create a widened channel; and
d) introducing an intracorneal implant into said widened channel through said incision.

2. The method of claim 1, wherein said widened channel comprises an annular channel having a width greater than the length of said incision.

3. The method of claim 1, wherein said widened channel comprises an intracorneal pocket having a width greater than the length of said incision.

4. The method of claim 1, wherein step b) comprises inserting a dissector blade through said incision and rotating the dissector blade through a circular path to form said circular intracorneal channel.

5. The method of claim 1, wherein step b) comprises the substeps of inserting a clockwise dissector blade through said incision and rotating the clockwise dissector blade clockwise to form a clockwise channel and inserting a counterclockwise dissector blade through said incision and rotating the counterclockwise dissector blade counterclockwise to form a counterclockwise channel.

6. The method of claim 1, wherein step c) comprises inserting a channel-widening dissector blade having a side leg through said incision and rotating the channel-widening dissector blade through said circular intracorneal channel to widen said circular intracorneal channel.

7. The method of claim 1, wherein step c) comprises the substeps of inserting a clockwise channel-widening dissector blade having a side leg through said incision and rotating the clockwise channel-widening dissector blade clockwise to widen said circular intracorneal channel and inserting a counterclockwise channel-widening dissector blade having a side leg through said incision and rotating the counterclockwise channel-widening dissector blade counterclockwise to widen said circular intracorneal channel.

8. The method of claim 1, wherein step c) comprises inserting a pocket-forming dissector blade having a side leg through said incision and rotating the pocket-forming dissector blade through said circular intracorneal channel to widen said circular intracorneal channel into an intracorneal pocket.

9. The method of claim 8, wherein said implant comprises an intracorneal lens, lenticule or inlay.

10. The method of claim 9, wherein said implant is folded.

11. The method of claim 8, wherein said implant has a central aperature.

12. The method of claim 1, wherein step c) comprises the substeps of inserting a clockwise pocket-forming dissector blade having a side leg through said incision and rotating the clockwise pocket-forming dissector blade clockwise to widen said circular intracorneal channel and inserting a counterclockwise pocket-forming dissector blade having a side leg through said incision and rotating the counterclockwise pocket-forming dissector blade counterclockwise to widen said circular intracorneal channel, thereby forming an intracorneal pocket.

13. The method of claim 1, wherein step c) comprises the substeps of inserting a channel-widening dissector blade having a side leg through said incision and rotating the channel-widening dissector blade through said circular intracorneal channel to widen said circular intracorneal channel and inserting a pocket-forming dissector blade having a longer side leg through said incision and rotating the pocket-forming dissector blade through said circular intracorneal channel to widen said circular intracorneal channel into an intracorneal pocket.

14. The method of claim 1, wherein step c) comprises inserting a dissector blade through said incision and dissecting a region of said cornea bounded by said circular intracorneal channel to create an intracorneal pocket.

15. The method of claim 1, wherein step d) comprises positioning said intracorneal implant within said intracorneal cavity at a location remote from said incision.

16. The method of claim 1, wherein step d) comprises introducing said intracorneal implant through said incision in a folded condition.

17. The method of claim 16, further comprising the step of e) unfolding said intracorneal implant within said intracorneal cavity.

18. A dissector for forming an intracorneal cavity, said dissector comprising an arc-shaped member having a distal end and support end, said distal end including a leg portion extending from said distal end.

19. A kit for forming an intracorneal cavity, said kit comprising:
a first dissector for forming a circular intracorneal channel;
a second dissector for widening said circular intracorneal channel to create an intracorneal cavity.

20. A method of preparing an intracorneal pocket comprising the steps of:
a) cutting a small incision in the anterior surface of the cornea of an eye;
b) creating a circular intracorneal channel originating at said incision;
c) widening said circular intracorneal channel to create a widened channel; and
d) dissecting radially inward from said widened channel until said pocket is formed.

21. A method of inserting an intracorneal continuous ring implant comprising the steps of
a) creating a small incision in said cornea;
b) forming an open pocket within said cornea through said incision; and
c) inserting a continuous ring implant into said open pocket through said incision.

22. The method of claim 18, wherein said continuous ring implant is inserted in a stretched state.

23. The method of claim 18, wherein said continuous ring implant is folded prior to insertion.

24. The method of claim 20, wherein said continuous ring implant is inserted into an arc-shaped tube prior to insertion into said open pocket.

25. An intracorneal insert for introduction into the cornea of a human eye, said insert having a continuous ring shape.
